(19) United States
(12) Patent Application Publication
(10) Pub. No.: US 2023/0165853 A1
(43) Pub. Date: Jun. 1, 2023

(54) METHOD FOR TREATING CHRONIC KIDNEY DISEASES

(71) Applicants: ENYO PHARMA, LYON (FR); INSERM (INSTITUT NATIONAL DE LA SANTE ET DE LA RECHERCHE MEDICALE), PARIS (FR)

(72) Inventors: FABIOLA TERZI, PARIS (FR); RAPHAËL DARTEIL, LYON (FR); PIETRO SCALFARO, FIESCH (CH); JACKY VONDERSCHER, SENOUILLAC (FR); HUGO GIRMA, CHABANIERE (FR)

(21) Appl. No.: 17/910,385

(22) PCT Filed: Jan. 13, 2022
(86) PCT No.: PCT/EP2022/050592

§ 371 Date: Sep. 9, 2022

(30) Foreign Application Priority Data
Jan. 14, 2021 (EP) 21305036.2
Oct. 20, 2021 (EP) 21306466.0

(51) Int. Cl. A61K 31/496; A61P 13/12; A61P 1/16

(52) U.S. Cl. CPC A61K 31/496; A61P 13/12; A61P 1/16

(57) ABSTRACT

The present invention relates to a method for treating chronic kidney diseases.

### CROSS-REFERENCE TO RELATED APPLICATION

This application is the U.S. national stage application of International Patent Application No. PCT/EP2022/050592, filed Jan. 13, 2022.

### FIELD OF THE INVENTION

The present invention relates to the field of the medicine, in particular of chronic kidney diseases (CKD).

### BACKGROUND OF THE INVENTION

Chronic kidney disease (CKD) is a long-term condition in which the kidneys do not work correctly. It has a high prevalence and is estimated to affects hundreds of millions of people. Diabetes and hypertension are the two principal causes of CKD. Although the kidney has a capacity for regeneration after acute injury, in the context of chronic injury, regeneration and recovery are much more difficult. Accordingly, this process is often irreversible, leading to end-stage renal disease, a situation that requires dialysis or renal transplantation.

The progression of CKD is characterized by the loss of nephrons, the functional units of kidney, and their replacement by extracellular matrix (ECM), independently of the associated disease. Thus, one of the consequences of CKD is glomerulosclerosis and tubulointerstitial fibrosis caused by an imbalance between excessive synthesis and reduced breakdown of the ECM. There are currently no effective treatments for preventing the progression of renal fibrosis. The presence of fibrosis in CKD is strongly related to the future manifestation of renal failure and has thus been related with poor long-term prognosis.

More precisely, progressive CKD may be viewed as having three phases. First, there is cause-specific injury and acute response to that injury. In the second phase, misdirected repair generates fibrosis and dysfunction. At this phase, although fibrosis is a pathological and destructive event, it is essentially a self limiting repair process to restrict the injury. The third and final stage is that of relatively steady progressive loss of remnant nephrons, which requires multiple nascent injury to each nephron or cluster of nephrons.

The farnesoid X receptor (FXR) is a bile acid (BA)-activated nuclear receptor highly expressed in the liver, gall bladder, intestines, and kidney that, upon activation, regulates bile acid production, conjugation, and transport. FXR initiates homeostatic responses to control bile acid levels by inducing genes involved in bile acid efflux, conjugation, detoxification, and renal excretion. FXR agonists have been suggested for the treatment of diabetic nephropathy. More specifically, Nidufexor (LMB763), a FXR agonist, is currently under investigation in Phase II clinical trials for the treatment of patients with NASH and diabetic nephropathy.

Number of patent applications disclosing FXR agonists suggest to use them for the treatment of renal diseases. For illustration, see non-exhaustively WO2009/127321, WO2020/231917, WO2020/168143, WO2020/150136.

Current therapies have limited effectiveness and only delay disease progression, underscoring the need to develop novel therapeutic approaches to either stop or reverse progression. Therefore, it is a strong unmet need of treatment in order to achieve a reversion/elimination of renal fibrosis.

### SUMMARY OF THE INVENTION

The inventors surprisingly identified compounds (Vonafexor and dEF2572) that are more efficient for treating a renal disease than two reference FXR agonists such as Nidufexor or Obeticholic acid. Indeed, even if the Nidufexor is a more powerful FXR agonist than the identified compounds in the kidney (FIG. 4), these compounds present a better efficiency for an unknown and unpredictable reason. The same differential effect has been observed in comparison to Obeticholic acid (FIG. 6). More specifically, compounds of the present invention are able to partially but significantly reverse interstitial fibrosis whereas no significant effect of Nidufexor or Obeticholic acid has been observed (FIGS. 1 and 6). The identified compounds, especially Vonafexor, displayed also higher beneficial effects than Losartan, the standard of care in CKD. The therapeutic benefit on kidney has been confirmed in a clinical trial with Vonafexor.

Accordingly, the present invention relates to 4-halogeno-5-[4-(2,6-dichloro-benzenesulfonyl)-piperazin-1-yl]-benzofuran-2-carboxylic acid or a pharmaceutically salt thereof or a pharmaceutical composition comprising it for use for the treatment of a renal disease.

The 4-halogeno-5-[4-(2,6-dichloro-benzenesulfonyl)-piperazin-1-yl]-benzofuran-2-carboxylic acid can be 4-bromo-5-[4-(2,6-dichloro-benzenesulfonyl)-piperazin-1-yl]-benzofuran-2-carboxylic acid or 4-chloro-5-[4-(2,6-dichloro-benzenesulfonyl)-piperazin-1-yl]-benzofuran-2-carboxylic acid or a pharmaceutically salt thereof.

Preferably, the renal disease is a chronic kidney disease (CKD).

Optionally, the subject to be treated suffers from a hypertension, type 2 diabetes, type 1 diabetes, obesity, Non-Alcoholic Steatohepatitis (NASH), ageing, infectious glomerulonephritis, focal segmental glomerulosclerosis, IgA nephropathy, minimal change glomerulopathy, membranous nephropathy, renal vasculitis, urinary tract obstruction, genetic alterations, autoimmune diseases such as systemic lupus erythematosus (SLE), and drug- or toxin-induced nephropathy. Optionally, the subject to be treated has a renal fibrosis, especially a tubulointerstitial fibrosis.

### BRIEF DESCRIPTION OF THE FIGURES

FIG. 1: Compound dEF2572 treatment significantly decreases renal interstitial fibrosis in Nx mice. Mice were subjected to a subtotal nephrectomy (Nx). Treatments were initiated 5 weeks after Nx for a duration of 3 weeks. Kidneys were collected at sacrifice, and fibrosis was evaluated by quantification of the picrosirius red (PSR) staining positive areas. p-values are indicated on the graph.

FIG. 2: Compound dEF2572 treatment significantly decreases renal tubular dilatation in Nx mice. Mice were subjected to a subtotal nephrectomy (Nx). Treatments were initiated 5 weeks after Nx for a duration of 3 weeks. Kidneys were collected at sacrifice, and tubular dilatation was evaluated by quantification of the Periodic acid-Schiff (PAS) staining positive areas. p-values are indicated on the graph.

FIG. 3: Compound dEF2572 treatment significantly decreases renal glomerular lesions in Nx mice. Mice were subjected to a subtotal nephrectomy (Nx). Treatments were initiated 5 weeks after Nx for a duration of 3 weeks. Kidneys were collected at sacrifice, and the degree of glomerular lesions was evaluated using a semi-quantitative scoring system. The results were expressed as the mean of the scores of 40-60 glomeruli per mouse. p-values are indicated on the graph.

FIG. 4: Both compound dEF2572 and Nidufexor treatments significantly increase the renal expression of two FXR target genes (OSTA and OSTB) in Nx mice. Mice were subjected to a subtotal nephrectomy (Nx). Treatments were initiated 5 weeks after Nx for a duration of 3 weeks. Kidneys were collected at sacrifice, and OSTA and OSTB mRNA levels were assessed by quantitative RT-PCR. p-values are indicated on the graphs.

FIG. 5: Chronic kidney disease classification based upon glomerular filtration rate and albuminuria. Data from: KDIGO. Summary of recommendation statements. Kidney Int 2013, 3 (suppl), 5; and National Kidney Foundation. K/DOQI clinical practice guidelines for chronic kidney disease: evaluation, classification, and stratification, Am J Kidney Dis 2002, 39 (Suppl 1): S1.

FIG. 6: Vonafexor treatment significantly decreases renal interstitial fibrosis in Nx mice. Mice were subjected to a subtotal nephrectomy (Nx). Treatments were initiated 5 weeks after Nx for a duration of 3 weeks. Kidneys were collected at sacrifice, and fibrosis was evaluated by quantification of the picrosirius red (PSR) staining positive areas. p-values are indicated on the graph.

### DETAILED DESCRIPTION OF THE INVENTION

The examples show, in an animal model, that the compounds of the invention partially reverse renal fibrosis, especially interstitial fibrosis, tubular dilatation and glomerular lesions, all effects being significant.

The present invention relates to 4-halogeno-5-[4-(2,6-dichloro-benzenesulfonyl)-piperazin-1-yl]-benzofuran-2-carboxylic acid or a pharmaceutically salt thereof or a pharmaceutical composition comprising it for use for the treatment of a renal disease in a subject. It further relates to the use of 4-halogeno-5-[4-(2,6-dichloro-benzenesulfonyl)-piperazin-1-yl]-benzofuran-2-carboxylic acid or a pharmaceutically salt thereof or a pharmaceutical composition comprising it for the manufacture of a drug for the treatment of a renal disease in a subject. It also relates to a method for treating a renal disease in a subject in need thereof, comprising administering a therapeutic amount of 4-halogeno-5-[4-(2,6-dichloro-benzenesulfonyl)-piperazin-1-yl]-benzofuran-2-carboxylic acid or a pharmaceutically salt thereof or a pharmaceutical composition comprising it.

Accordingly, the compound for use according to the present invention is 4-halogeno-5-[4-(2,6-dichloro-benzenesulfonyl)-piperazin-1-yl]-benzofuran-2-carboxylic acid or a pharmaceutically salt thereof. More specifically, the compound can be 4-bromo-5-[4-(2,6-dichloro-benzenesulfonyl)-piperazin-1-yl]-benzofuran-2-carboxylic acid (dEF2572) or a pharmaceutically salt thereof. Alternatively, the compound can be 4-chloro-5-[4-(2,6-dichloro-benzenesulfonyl)-piperazin-1-yl]-benzofuran-2-carboxylic acid (EYP001) or a pharmaceutically salt thereof.

The compound 4-chloro-5-[4-(2,6-dichloro-benzenesulfonyl)-piperazin-1-yl]-benzofuran-2-carboxylic acid is also called EYP001 or Vonafexor (CAS No 1192171-69-9). Its structure is the following:

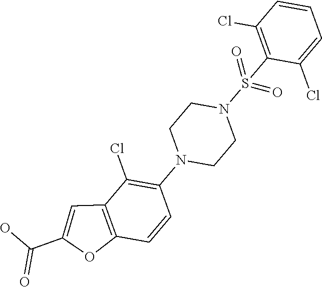 [SMILES: O=C(O)c1cc2c(Cl)c(N3CCN(S(=O)(=O)c4c(Cl)cccc4Cl)CC3)ccc2o1]

The compound 4-bromo-5-[4-(2,6-dichloro-benzenesulfonyl)-piperazin-1-yl]-benzofuran-2-carboxylic acid (CAS No 1192171-67-7) is also called dEF2572 and has the following structure:

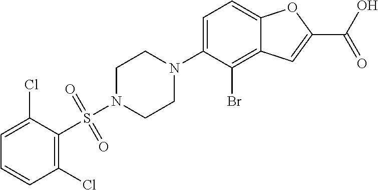 [SMILES: O=C(O)c1cc2c(Br)c(N3CCN(S(=O)(=O)c4c(Cl)cccc4Cl)CC3)ccc2o1]

### Definitions

According to the present invention, the terms below have the following meanings:

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

Renal Disease

In a particular aspect, the compounds of the present invention are able to decrease the renal fibrosis. For example, the decrease could be of 20, 30, 40, 50, 60, 70, 80, 90 or 100% in comparison of the renal fibrosis in absence of treatment with the compound. The decrease can be measured by any method available for the person skilled in the art, for instance in an animal model as detailed in the example. Optionally, the renal fibrosis is a glomerulosclerosis. Optionally, the renal fibrosis is more particularly a glomerulosclerosis. Optionally, the renal fibrosis is more particularly a tubulointerstitial fibrosis. Optionally, the renal fibrosis is both glomerulosclerosis and tubulointerstitial fibrosis.

In addition, the compounds of the present invention are able to decrease the tubular dilatation. For example, the decrease could be of 10, 20, 30, 40, 50, 60, 70, 80, 90 or 100% in comparison of the tubular dilatation in absence of treatment with the compound. The decrease can be measured by any method available for the person skilled in the art, for instance in an animal model as detailed in the example.

The compounds of the present invention are able to decrease glomerular lesions. For example, the decrease could be of 10, 20, 30, 40, 50, 60, 70, 80, 90 or 100% in comparison of the glomerular lesions in absence of treatment with the compound. The decrease can be measured by any method available for the person skilled in the art, for instance in an animal model as detailed in the example.

In one aspect, the subject to be treated has a renal fibrosis. The renal fibrosis can be diagnosed based on a kidney biopsy. Alternatively, it can be diagnosed based on an alternative analysis such as magnetic resonance imaging (MRI) or urinary tract proteomics (e.g., CKD273)

In a particular aspect, the renal fibrosis can include glomerulosclerosis. In another particular aspect, the renal fibrosis can include tubulointerstitial fibrosis. In an additional particular aspect, the renal fibrosis can include both glomerulosclerosis and tubulointerstitial fibrosis.

In a particular aspect, the renal disease is a chronic kidney disease. Preferably, the subject has a renal fibrosis and suffers from a chronic kidney disease.

Chronic Kidney Disease (CKD) is defined as the presence of kidney damage (usually detected as urinary albumin excretion of ≥30 mg/day or equivalent) or decreased kidney function (defined as estimated glomerular filtration rate [eGFR]<60 mL/min/1.73 m2) for three or more months, irrespective of the cause.

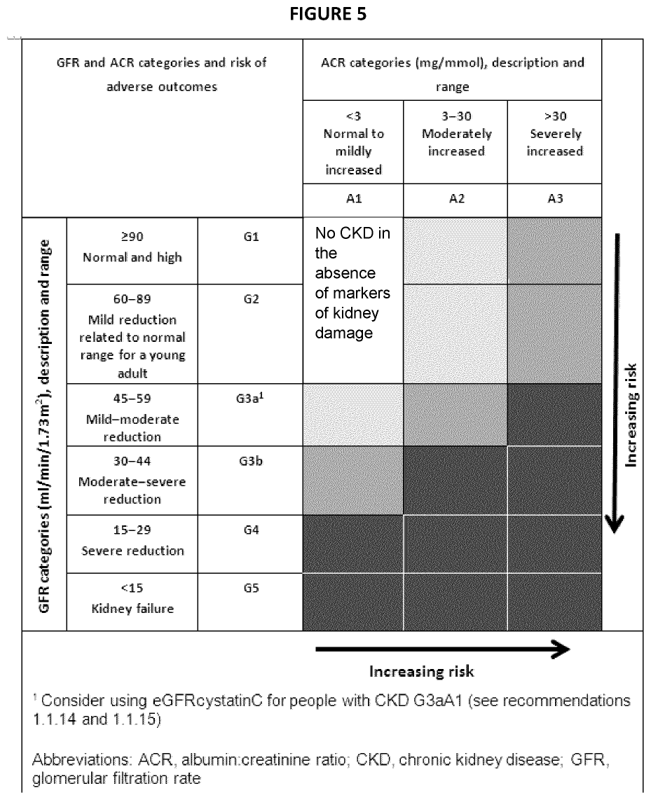

In a particular aspect, the CKD is a CKD with a stage chosen from G1, G2, G3a, G3b, G4 or G5, preferably G1, G2, G3a, G3b, or G4, based upon glomerular filtration rate (eGFR) as indicated in FIG. 5, more preferably G2, G3a, G3b, or G4, still more particularly G2, G3a, or G3b. Preferably, the subject has a renal fibrosis.

In another particular aspect, the CKD is a CKD with a CKD stage chosen from A1, A2 or A3 based upon albuminuria (ACR) as indicated in FIG. 5.

TABLE 1
Staging of CKD

| | | ACR | | |
|---|---|---|---|---|
| Staging | | A1 | A2 | A3 |
| GFR | G1 | 1* if kidney | 1 | 2 |
| | | damage present | | |
| | G2 | 1* if kidney | 1 | 2 |
| | | damage present | | |
| | G3a | 1 | 2 | 3 |
| | G3b | 2 | 3 | 3 |
| | G4 | 3 | 4+ | 4+ |
| | G5 | 4+ | 4+ | 4+ |

Optionally, the CKD has a stage selecting from stage 1*, stage 1, stage 2 or stage 3 as defined in Table 1. Optionally, the CKD has a stage selecting from stage 1, stage 2 or stage 3 as defined in Table 1. Optionally, the CKD has a stage selecting from stage 1 or stage 2 as defined in Table 1.

In a first particular aspect, the CKD is a CKD of stage 1* defined in Table 1 or of G1 or G2 based upon eGFR as indicated in FIG. 5 and of A1 based upon ACR as indicated in FIG. 5.

In a second particular aspect, the CKD is a CKD of stage 1 defined in Table 1 or of G1 or G2 based upon eGFR as indicated in FIG. 5 and of A2 based upon ACR as indicated in FIG. 5 or of G3a based upon eGFR and of A1 based upon ACR.

In a third particular aspect, the CKD is a CKD of stage 2 defined in Table 1 or of G1 or G2 based upon eGFR as indicated in FIG. 5 and of A3 based upon ACR as indicated in FIG. 5 or of G3a based upon eGFR and of A2 based upon ACR, or of G3b based upon eGFR and of A1 based upon ACR.

In a fourth particular aspect, the CKD is a CKD of stage 3 defined in Table 1 or of G3a based upon eGFR as indicated in FIG. 5 and of A3 based upon ACR as indicated in FIG. 5 or of G3b based upon eGFR and of A2 or A3 based upon ACR, or of G4 based upon eGFR and of A1 based upon ACR.

The effect of the compound on the disease can be for instance assessed by the measure of eGFR or ACR. A therapeutic effect could be the improvement of eGFR and/or ACR. The therapeutic effect could be the stabilization of eGFR and/or ACR. The therapeutic effect could also be a delay of the progression of the disease or a slow-down of the progression of the disease, for instance as assessed by eGFR and/or ACR assays.

In a particular aspect, the subject has a renal fibrosis and suffers from a disease selected from the group consisting of hypertension, type 2 diabetes, type 1 diabetes, obesity, Non-Alcoholic Steatohepatitis (NASH), ageing, infectious glomerulonephritis, in particular infections such as syphilis, malaria, hepatitis B, hepatitis C or HIV, focal segmental glomerulosclerosis, IgA nephropathy, minimal change glomerulopathy, membranous nephropathy, renal vasculitis, urinary tract obstruction, genetic alterations, autoimmune diseases such as systemic lupus erythematosus (SLE), and drug- or toxin-induced nephropathy such as nephropathy induced by drugs such as captopril, NSAIDs, penicillamine, probenecid, bucillamine, anti-TNF therapy, and tiopronin or by toxins such as inorganic salts (e.g., gold, mercury).

Optionally, the subject has a renal fibrosis and suffers from a primary kidney disease such as focal segmental glomerulosclerosis, IgA nephropathy, minimal change glomerulopathy, membranous nephropathy, urinary-tract infections, stones, obstruction diseases, and renal vasculitis. Optionally, the subject has a tubulointerstitial fibrosis.

Optionally, the subject has a CKD, in particular of any particular stage as defined above, and suffers from a primary kidney disease such as focal segmental glomerulosclerosis, IgA nephropathy, minimal change glomerulopathy, membranous nephropathy, urinary-tract infections, stones, obstruction diseases, and renal vasculitis. Optionally, the subject has a renal fibrosis, especially a tubulointerstitial fibrosis.

In another particular aspect, the subject has a renal fibrosis and suffers from a systemic disease affecting the kidney, for instance a disease selected from the group consisting of hypertension, type 2 diabetes, type 1 diabetes, Non-Alcoholic Steatohepatitis (NASH), infectious glomerulonephritis, in particular infections such as syphilis, malaria, hepatitis B, hepatitis C or HIV, renal vasculitis, autoimmune diseases such as systemic lupus erythematosus (SLE), and drug- or toxin-induced nephropathy such as neuphropathy induced by drugs such as captopril, NSAIDs, penicillamine, probenecid, bucillamine, anti-TNF therapy, and tiopronin or by toxins such as inorganic salts (e.g., gold, mercury). Optionally, the subject has a tubulointerstitial fibrosis.

Optionally, the subject has a CKD, in particular of any particular stage as defined above, and suffers from a systemic disease affecting the kidney, for instance a disease selected from the group consisting of hypertension, type 2 diabetes, type 1 diabetes, Non-Alcoholic Steatohepatitis (NASH), infectious glomerulonephritis, in particular infections such as syphilis, malaria, hepatitis B, hepatitis C or HIV, renal vasculitis, autoimmune diseases such as systemic lupus erythematosus (SLE), and drug- or toxin-induced nephropathy such as neuphropathy induced by drugs such as captopril, NSAIDs, penicillamine, probenecid, bucillamine, anti-TNF therapy, and tiopronin or by toxins such as inorganic salts (e.g., gold, mercury). Optionally, the subject has a renal fibrosis, especially a tubulointerstitial fibrosis.

Optionally, the subject suffers from hypertension. The subject may have a CKD of stage 1, 2 or 3 as defined above in Table 1. Optionally, the subject has a renal fibrosis, especially a tubulointerstitial fibrosis.

Optionally, the subject suffers from type 2 diabetes or type 1 diabetes. The subject may have a CKD of stage 1, 2 or 3 as defined above in Table 1. Optionally, the subject has a renal fibrosis, especially a tubulointerstitial fibrosis.

Optionally, the subject suffers from systemic lupus erythematosus (SLE). The subject may have a CKD of stage 1, 2 or 3 as defined above in Table 1. In this context, the subject may have a lupus nephropathy of class III, IV, V or VI. Optionally, the subject has a renal fibrosis, especially a tubulointerstitial fibrosis.

Optionally, the subject suffers from NASH or NAFLD. The subject may have a CKD of stage 1, 2 or 3 as defined above defined in Table 1. Non-alcoholic steatohepatitis (NASH) is a disease characterized by excessive fat accumulation, inflammation, and ballooning degeneration of hepatocytes, with or without fibrosis in the liver. In addition, some subjects affected by NASH may further present chronic kidney disease. For these particular subjects, the compounds of the invention could be of particular interest. Indeed, the compounds of the invention are able to decrease significantly the inflammation and fibrosis in a dose-dependent manner in the liver and they are further capable of significantly inhibiting fibrosis in the kidney and even reversing the existing fibrosis and also of decreasing renal inflammation. Accordingly, the compounds of the present invention could be useful for protecting a subject suffering from NASH of liver and renal lesions or for treating a subject suffering from NASH so as to limit, slow down or reverse liver and renal lesions. Then, the subject is in particular a subject suffering from NASH or NAFLD and having a renal fibrosis. Optionally, the subject is a subject suffering from NASH or NAFLD and from CKD. Optionally, the subject has a renal fibrosis, especially a tubulointerstitial fibrosis.

Optionally, the renal disease is selected from the group consisting of AIDS-associated nephropathy, ischemic nephropathy, tubulointerstitial nephropathy, hepatorenal syndrome, hydronephrosis, renal dysplasia, medullary cystic kidney disease, medullary sponge kidney, multicystic dysplastic kidney, podocytopathy, kidney papillary necrosis, nephritis including glomerulonephritis, hereditary nephritis, interstitial nephritis, pyelitis, nephrocalcinosis, nephrosclerosis, Alport's syndrome, Fabry's disease, renal sarcoidosis, diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease.

Optionally, the renal disease is selected from the group consisting of AIDS-associated nephropathy, ischemic nephropathy, tubulointerstitial nephropathy, hepatorenal syndrome, hydronephrosis, renal dysplasia, medullary cystic kidney disease, medullary sponge kidney, multicystic dysplastic kidney, podocytopathy, kidney papillary necrosis, nephritis including glomerulonephritis, hereditary nephritis, interstitial nephritis, pyelitis, nephrocalcinosis, nephrosclerosis, Alport's syndrome, Fabry's disease and renal sarcoidosis.

Optionally, the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease.

In a particular aspect, the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease and the compound is 4-bromo-5-[4-(2,6-dichloro-benzenesulfonyl)-piperazin-1-yl]-benzofuran-2-carboxylic acid or a pharmaceutically salt thereof.

In another particular aspect, the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease and the compound is to be administered two or three times a day.

Combinations

The compounds of the present disclosure can be used in combination with other therapeutic agents. The additional therapeutic agents can be selected from the agents already used for the treatment of one of the diseases as specified above. In particular, the additional therapeutic agent could be an anti-inflammatory agent.

Pharmaceutical Composition

The pharmaceutical composition comprises a compound of the present invention and optionally at least one pharmaceutically acceptable carrier or excipient.

The compound according to the present disclosure or the pharmaceutical composition according to the present disclosure may be administered by any conventional route of administration. In particular, the compound or the pharmaceutical composition of the present disclosure can be administered by a topical, enteral, oral, parenteral, intranasal, intravenous, intra-arterial, intramuscular, subcutaneous or intraocular administration and the like.

In particular, the compound according to the present disclosure or the pharmaceutical composition according to the present disclosure can be formulated for a topical, enteral, oral, parenteral, intranasal, intravenous, intra-arterial, intramuscular, subcutaneous or intraocular administration and the like.

Preferably, the compound according to the invention or the pharmaceutical composition according to the present disclosure is administered by enteral or parenteral route of administration. When administered parenterally, the compound according to the present disclosure or the pharmaceutical composition according to the present disclosure is preferably administered by intravenous route of administration. When administered enterally, the compound according to the present disclosure or the pharmaceutical composition according to the present disclosure is preferably administered by oral route of administration.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

The compound according to the invention or the pharmaceutical composition according to the present disclosure may be administered as a single dose or in multiple doses.

Preferably, the treatment is administered regularly, preferably between every day and every month, more preferably between every day and every two weeks, more preferably between every day and every week, even more preferably the treatment is administered every day.

In a particular embodiment, the treatment is administered daily, optionally 1, 2 or 3 times a day. In a particular aspect, the treatment is administered is administered at least twice a day, for particularly 2 or 3 times a day. In an alternative aspect, the treatment is administered is administered once a day.

The duration of treatment with the compound according to the invention or the pharmaceutical composition according to the invention can be weeks, months or even years. In particular, the duration of treatment may last as long as the disease persists.

The amount of compound according to the present disclosure or of pharmaceutical composition according to the present disclosure to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a particular aspect, the total compound dose for each administration of the compound according to the present disclosure or of the pharmaceutical composition according to the present disclosure is comprised between 0.00001 and 1 g.

The form of the pharmaceutical compositions, the route of administration and the dose of administration of the compound according to the present disclosure, or the pharmaceutical composition according to the present disclosure can be adjusted by those skilled in the art according to the type and severity of the disease, and to the patient, in particular its age, weight, sex, and general physical condition.

Further aspects and advantages of the present invention will be described in the following examples, which should be regarded as illustrative and not limiting.

### EXAMPLES

### Example 1

Materials and Methods

Experimental Plan

Analysis has been focused on an experimental model of CKD, i.e. the subtotal nephrectomy (Nx). In this model, a “curative study” was investigated, i.e. treatments have been initiated 5 weeks after Nx, when lesions are already developed. 42 nine-week old FVB female mice have been investigated, of which 6 have been submitted to sham operation (controls) and 36 to subtotal nephrectomy (Nx), which consist in the excision of the right kidney and the two poles of the left kidney to reach 75% reduction of total renal mass. Surgery was performed under xylazine (Rompun 2%; Bayer, Leverkusen, France) (6 μg/g of body weight) and ketamine (Clorketam 1000; Vetoquinol SA, Lirre, France) (120 μg/g of body weight) anesthesia.

After 5 weeks, mice have been divided into 4 groups:
Sham mice treated with the vehicle (n=6)
Nx mice treated with the vehicle (n=10)
Nx mice treated with dEF2572 FXR agonist (4-bromo-5-[4-(2,6-dichloro-benzenesulfonyl)-piperazin-1-yl]-benzofuran-2-carboxylic acid) at 100 mg/Kg/day (n=11)
Nx mice treated with Nidufexor (LMB763) FXR agonist at 30 mg/Kg/day (n=10)

Compounds have been administered once a day (QD) per oral gavage.

Mice have been sacrificed 3 weeks later. Blood has been collected just before sacrifice. At sacrifice, kidneys have been harvested for morphological and mRNA analyses.

Histological Methods

Kidneys were fixed in 4% paraformaldehyde, paraffin embedded, and 4-μm sections were stained with Periodic acid-Schiff (PAS), Masson's trichrome, Hematoxylin and eosin, and picrosirius red (PSR). Images were acquired using a Nikon Digital Camera Dx/m/1200. All sections were evaluated by a pathologist who was blinded to the treatment groups.

The degree of glomerular lesions was evaluated on Periodic acid-Schiff (PAS) staining using a semi-quantitative score methodology. Briefly, 40-60 glomeruli per mouse were scored at a magnification of ×400, using the following scoring system: 0=no lesion, 1=mild sclerosis, interesting 10-25% of the glomerulus, 2=moderate sclerosis, interesting 25%-50% of the glomerulus, 3=severe sclerosis interesting 50%-75% of the glomerulus, and 4=global sclerosis interesting >75% of the glomerulus. The results were expressed as the mean of the scores of 40-60 glomeruli per mouse.

The degree of tubular dilations was automatically quantified on PAS staining using Image J/Fiji software, version 2.1.0. The extent of interstitial fibrosis was also quantified using Image J/Fiji software. Staining of all kidney samples with picrosirius red (PSR) was performed simultaneously, and red intensity above a defined threshold was defined as fibrosis. For tubular dilations and interstitial fibrosis, at least 10 to 15 random selected fields (magnification ×200) across the corticomedullary junction were analyzed per each kidney section, and the results were expressed as percentages of the total area of the selected fields.

Results

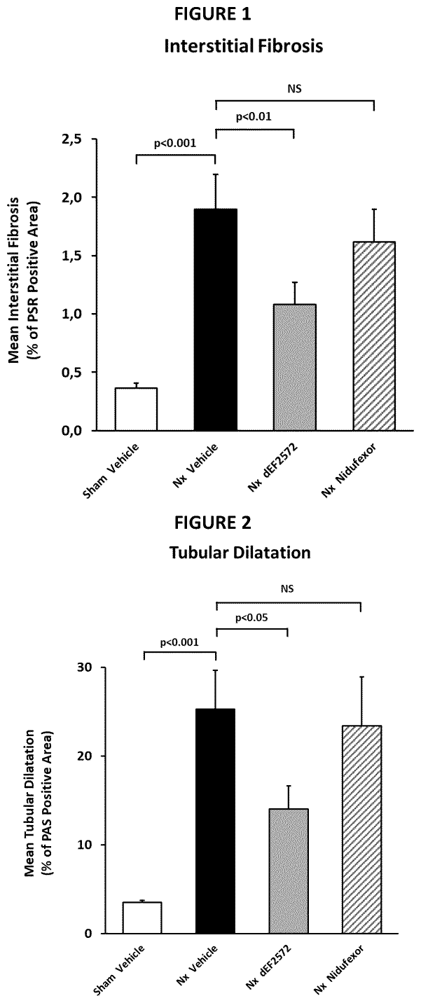

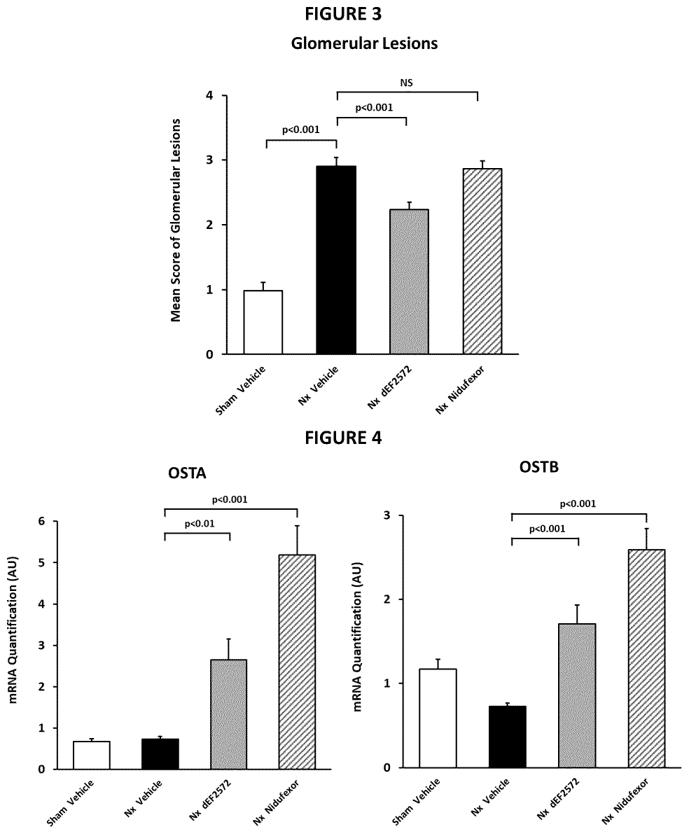

Two FXR agonists, compound dEF2572 (4-bromo-5-[4-(2,6-dichloro-benzenesulfonyl)-piperazin-1-yl]-benzofuran-2-carboxylic acid) and Nidufexor (also called LMB763) were evaluated in a mouse model of Chronic Kidney Disease (CKD). Mice were subjected to a subtotal nephrectomy (Nx), and 5 weeks after disease induction, treatments were initiated for a duration of 3 weeks. Kidneys were collected at sacrifice to assess renal lesions by histopathology. As shown on FIGS. 1, 2 and 3, compound dEF2572 treatment significantly decreases renal interstitial fibrosis, tubular dilatation and glomerular lesions respectively, in Nx mice.

It should be noted that those beneficial effects on the kidneys, induced by compound dEF2572, were not observed upon treatment with Nidufexor, despite the strong renal FXR target engagement of both compounds, as shown by the induction of OSTA and OSTB gene expression in kidneys (FIG. 4).

### Example 2

Materials and Methods

Experimental Plan

Analysis has been focused on an experimental model of CKD, i.e. the subtotal nephrectomy (Nx). In this model, a “curative study” was investigated, i.e. treatments have been initiated 5 weeks after Nx, when lesions are already developed.

54 nine-week old FVB female mice have been investigated, of which 6 have been submitted to sham operation (controls) and 48 to subtotal nephrectomy (Nx), which consist in the excision of the right kidney and the two poles of the left kidney to reach 75% reduction of total renal mass. Surgery was performed under xylazine (Rompun 2%; Bayer, Leverkusen, France) (6 μg/g of body weight) and ketamine (Clorketam 1000; Vetoquinol SA, Lirre, France) (120 μg/g of body weight) anesthesia.

After 5 weeks, mice have been divided into 5 groups:
Sham mice treated with the vehicle (n=6)
Nx mice treated with the vehicle (n=9)
Nx mice treated with Vonafexor FXR agonist at 100 mg/Kg/day (n=12)
Nx mice treated with Obeticholic acid (OCA) FXR agonist at 30 mg/Kg/day (n=12)
Nx mice treated with Losartan at 30 mg/Kg/day (n=12)

Compounds have been administered once a day (QD) per oral gavage.

Mice have been sacrificed 3 weeks later. At sacrifice, kidneys have been harvested for morphological analyses.

Histological Methods

Kidneys were fixed in 4% paraformaldehyde, paraffin embedded, and 4-μm sections were stained with picrosirius red (PSR). Images were acquired using a Nikon Digital Camera Dx/m/1200. All sections were evaluated by a pathologist who was blinded to the treatment groups.

Staining of all kidney samples with picrosirius red (PSR) was performed, and red intensity above a defined threshold was defined as fibrosis. For interstitial fibrosis, at least 10 to 15 random selected fields (magnification ×200) across the corticomedullary junction were analyzed per each kidney section, and the results were expressed as percentages of the total area of the selected fields.

Results

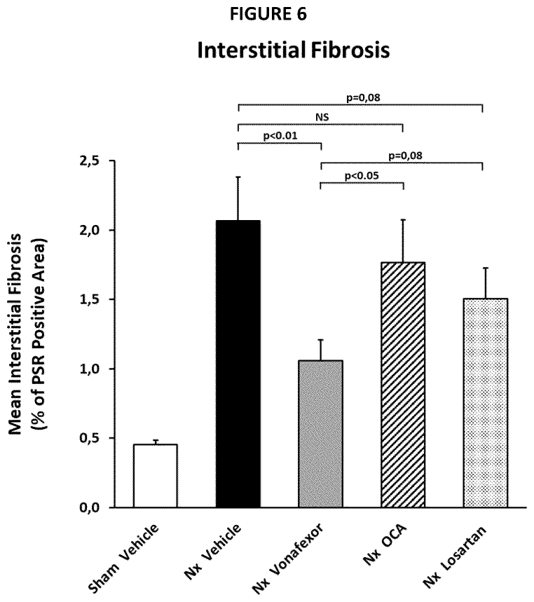

Two FXR agonists, Vonafexor and OCA, and Losartan, were evaluated in a mouse model of Chronic Kidney Disease (CKD). Mice were subjected to a subtotal nephrectomy (Nx), and 5 weeks after disease induction, treatments were initiated for a duration of 3 weeks. Kidneys were collected at sacrifice to assess interstitial fibrosis by histopathology. As shown on FIG. 6, Vonafexor treatment significantly decreases renal interstitial fibrosis in Nx mice.

It should be noted that those beneficial effects on the kidneys, induced by Vonafexor, were not observed upon treatment with OCA, Vonafexor being significantly better than OCA. Moreover, Vonafexor displayed also higher beneficial effects than Losartan, the standard of care in CKD.

### Example 3

Vonafexor Induced Hepatic and Renal Improvement in the Randomized, Doubled-Blind, Placebo-Controlled Livify NASH Trial

Vonafexor has anti-fibrotic effects in NASH and Chronic Kidney Disease (CKD) models. Up to 64% of NASH patients have decreased estimated glomerular filtration rate (eGFR <90 mL/min/1.73 m²) which can evolve into CKD. Phase 2a results in NASH patients with normal or mildly decreased eGFR treated are reported below.

Methods

96 patients were randomized 1:1:1 to daily oral Placebo (PBO, n=32) or VONA (Vonafexor) 100 mg (n=31) or 200 mg (n=33) for 12 weeks (W12). Inclusion criteria required phenotypic stage 2 or 3 fibrosis NASH with an absolute liver fat content (LFC by MRI-PDFF) ≥10% and liver stiffness by transient elastography [LSTE]≥8.5 kPa or previous biopsy-proven NASH. Randomization was stratified by diabetes and LFC.

Results

Baseline characteristics were similar between arms. There was a statistically significant reduction in absolute LFC (liver fatty content) at W12 in VONA-treated patients (−6.3% with 100 mg, −5.5% with 200 mg, and −2.3% with PBO, p<0.001) (Table 2). Absolute LFC reduction of >5% was achieved in 58% of patients in the 100 mg group vs 22% in the PBO group, and relative LFC reduction of >30% was achieved in 50% of VONA-treated vs 13% of PBO patients.

VONA treatment achieved a significant 26% mean reduction in ALT vs 13% for PBO. A rapid and sustained 42% mean reduction in GGT was also observed in VONA-treated subjects (p<0.001). The liver fibro-inflammation marker cT1 (Liver Multiscan) was reduced by 81 msec in the 100 mg VONA arm compared to 10 msec in the PBO arm (p<0.001).

A 34% increase in low density lipoprotein-cholesterol (LDL-C) was observed. Statin dose adjustment normalized LDL-C levels to 70 mg/dL. 9% of patients discontinued participation with VONA 100 mg due to pruritus, which was mostly mild, transient and localized. No ALT increases Grade 2 were reported. Five non-drug related severe adverse events were reported (1 in the PBO group and 2 in each of the VONA 100 mg and 200 mg groups).

For the renal aspect, a significant mean improvement in eGFR [+5.6 mL/min/1.73 m²] was observed in VONA 100 mg or 200 mg treated subjects, while a decrease in eGFR [−2.8 mL/min/1.73 m2] was observed in the PBO group. Over the 12-week treatment period and in VONA 100 mg treated subjects, 76% of the patients receiving VONA had an eGFR increase >0.1 mL/min/1.73 m, while 66% of patients receiving placebo had a decrease of their kidney function.

VONA treatment shows a significant benefit on eGFR and also reduced alpha-2-Macroglobulin (A2M) plasma levels. A2M and eGFR are parameter in the assessment of renal disease. A2M is a major human plasma protein with various functions including ion transport, carrier protein, and the inhibition of proteinases. Elevated levels are seen in clinical conditions such as cirrhosis and liver fibrosis, nephrotic syndrome, severe burns, osteonecrosis of the femoral head and diabetes. Levels of A2M correlated with the eGFR in diabetic patients who have CKD but not with glycemia control parameters. These patients had increased A2M levels and the urinary albumin:creatinine ratio (AcR) correlated with levels of A2M. Surprisingly the FXR agonism by Vonafexor reduced A2M plasma levels in NASH patients and this reduction correlated with the eGFR improvement (p<0.05). A2M decrease can be considered as a biomarker for an improved renal function due to Vonafexor and used to monitor treatment response to Vonafexor.

Taken together and surprisingly, in addition to favorable liver effects, a renal benefit is associated with a treatment with VONA. Overall VONA was safe and well tolerated, with the 100 mg dose showing a more favorable tolerability-efficacy profile.

TABLE 2
Baseline values and change from baseline to W12 in key study outcomes

| | Placebo | VONA 100 mg | VONA 200 mg |
|---|---|---|---|
| | (n = 32) | (n = 31) | (n = 33) |
| Liver fat content (LFC) | | | |
| Baseline LFC, % | 20.9 (7.2) | 19.8 (6.3) | 20.1 (6.7) |
| Change Absolute LFC, % | −2.3 (0.9) | −6.3 (0.9) #, § | −5.5 (0.9) #, § |
| % Patients with >5% absolute LFC reduction | 22% | 57 % § | 43% § |
| Change Relative LFC, % | −10.5 (4.3) | −30.0 (4.7) #, § | −25.5 (4.6) #, § |
| % Patients with >30% absolute reduction | 13% | 50% § | 39% § |
| Non-invasive NASH Biomarkers | | | |
| Baseline ALT (IU/L) | 51.7 (28.0) | 53.4 (29.9) | 49.8 (27.7) |
| ALT, absolute change (IU/L) | −11.5 (3.2) | −19.3 (3.2) § | −10.8 (3.3) |
| % Patients with >−17 IU/L reduction | 25% | 52% § | 36% |
| GGT, absolute change (IU/L) | −3.9 (3.1) # | −40.6 (3.3) #, § | −34.1 (3.3) #, § |
| Alpha-2-Macroglobulin, absolute change | +4.3 (3.8) # | −25.9 (4.3) #, § | −18.4 (4.5) #, § |
| (mg/dL) | | | |
| Baseline fibro-inflammation marker cT1 (msec) | 903 (95) | 874 (128) | 925 (99) |
| cT1, absolute change (msec) | −10.2 (14.5) | −80.6 (15.4) #, § | −71.9 (14.2) #, § |
| % Patients >88 msec absolute reduction | 12% | 36% * | 36% * |
| Clinical Biomarkers | | | |
| Baseline BMI | 34.3 (4.3) | 34.3 (4.1) | 35.4 (5.1) |
| Body Weight, absolute change (kg) | −0.1 (0.5) | −1.7 (0.5) #,§ | −2.5 (0.5) #, § |
| Waist-to-Hip Ratio, absolute change | 0.01 (0.0) | −0.02 (0.0) #, § | −0.01 (0.0) |
| Baseline eGFR (mL/min/1.73 m²) | 91.4 (18.7) | 86.4 (16.4) | 93.0 (21.7) |
| eGFR, absolute change (mL/min/1.73 m²) | −2.9 (14.2) | +6.0 (9.5) #, § | +2.5 (14.5) #, § |
| % Patients with any eGFR increase | 34% | 76% § | 65% § |

Shown are mean (SD) or % patients at baseline and changes at W12. LFC measured by MRI-PDFF.
# p < 0.05 vs baseline,
§ p < 0.05 vs Placebo,
* p = 0.06

1-15. (canceled)

16. A method of treating a subject having a renal disease comprising administering a therapeutic amount of a 4-halogeno-5-[4-(2,6-dichloro-benzenesulfonyl)-piperazin-1-yl]-benzofuran-2-carboxylic acid compound or a pharmaceutically salt thereof to said subject.

17. The method according to claim 16, wherein the compound is 4-bromo-5-[4-(2,6-dichloro-benzenesulfonyl)-piperazin-1-yl]-benzofuran-2-carboxylic acid or a pharmaceutically salt thereof.

18. The method according to claim 16, wherein the compound is 4-chloro-5-[4-(2,6-dichloro-benzenesulfonyl)-piperazin-1-yl]-benzofuran-2-carboxylic acid or a pharmaceutically salt thereof.

19. The method according to claim 16, wherein the renal disease is a chronic kidney disease (CKD).

20. The method according to claim 19, wherein the CKD has a stage selected from stage 1*, stage 1, stage 2 or stage 3 as defined in Table 1.

21. The method according to claim 19, wherein the CKD has a stage selected from stage 1, stage 2 or stage 3 as defined in Table 1.

22. The method according to claim 19, wherein the CKD has a stage selected from stage 1 or stage 2 as defined in Table 1.

23. The method according to claim 16, wherein the subject suffers from a hypertension, type 2 diabetes, type 1 diabetes, obesity, Non-Alcoholic Steatohepatitis (NASH), ageing, infectious glomerulonephritis, focal segmental glomerulosclerosis, IgA nephropathy, minimal change glomerulopathy, membranous nephropathy, renal vasculitis, urinary tract obstruction, genetic alterations, autoimmune diseases, systemic lupus erythematosus (SLE), drug-induced nephrotoxixity or toxin-induced nephropathy.

24. The method according to claim 16, wherein the subject has a renal fibrosis or a tubulointerstitial fibrosis.

25. The method according to claim 16, wherein the renal disease is selected from the group consisting of AIDS-associated nephropathy, ischemic nephropathy, tubulointerstitial nephropathy, hepatorenal syndrome, hydronephrosis, renal dysplasia, medullary cystic kidney disease, medullary sponge kidney, multicystic dysplastic kidney, podocytopathy, kidney papillary necrosis, nephritis, glomerulonephritis, hereditary nephritis, interstitial nephritis, pyelitis, nephrocalcinosis, nephrosclerosis, Alport's syndrome, Fabry's disease and renal sarcoidosis.

26. The method according to claim 16, wherein the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease.

27. The method according to claim 16, wherein the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease and the compound is 4-bromo-5-[4-(2,6-dichloro-benzenesulfonyl)-piperazin-1-yl]-benzofuran-2-carboxylic acid or a pharmaceutically salt thereof.

28. The method according to claim 16, wherein the renal disease is selected from the group consisting of diabetic nephropathy, focal segmental glomerulosclerosis (FSGS), hypertensive nephrosclerosis, chronic glomerulonephritis, chronic transplant glomerulopathy, chronic interstitial nephritis, Sjogren's syndrome, Alagille syndrome, alpha 1-antitrypsin deficiency, and polycystic kidney disease and the compound is administered two or three times a day.

29. The method according to claim 16, wherein the compound is administered two or three times a day.

30. The method according to claim 16, wherein the compound is administered once a day.
